SEQUENCE LISTING (7 sequences)

SEQ ID NO: 1 - protein, 299 aa, Escherichia coli
  misc_feature: Wild type SLT-1 A chain
IEGRASKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVARMASDEFPSMCPADGRVRGITHNKILWDSSTLGAILMRRTISS

SEQ ID NO: 2 - DNA, 32 bp, Artificial
  Primer
gttactgtgacagctgaagctttacgttttcg

SEQ ID NO: 3 - DNA, 31 bp, Artificial
  Primer
gagaagaagagactgcagattccatctgttg

SEQ ID NO: 4 - protein, 302 aa, Artificial
  SLT-1 A Chain lib#3 protein sequence (SAM3)
KGMRSHHHHHHHHIEGRASKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHIYSNKLMASRVARMASDEFPSMCPADGRVRGITHNKIL

SEQ ID NO: 5 - protein, 319 aa, Artificial
  SLT-1 A Chain lib#5 protein sequence (SAM5)
KGMRSHHHHHHHHIEGRASKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHAAFADLIASRVARMASDEFPSMCPADGRVRGITHNKILWDSSTLGAILMRRTISS

SEQ ID NO: 6 - protein, 7 aa, Artificial
  First melanoma active insert
IYSNKLM

SEQ ID NO: 7 - protein, 7 aa, Artificial
  Second melanoma active insert
AAFADLI

(19) United States
(12) Patent Application Publication
(10) Pub. No.: US 2007/0298434 A1
(43) Pub. Date: Dec. 27, 2007

(54) Library of Toxin Mutants, and Methods of Using Same

(76) Inventors: Jean Gariepy, Toronto (CA); Xin Wei, Toronto (CA)

Correspondence Address: Marina Larson & Associates, LLC, P.O. BOX 4928, DILLON, CO, 80435, US

(21) Appl. No.: 10/598,965

(22) PCT Filed: Mar. 26, 2004
(86) PCT No.: PCT/CA04/00443

§ 371 Date: Feb. 26, 2007

(51) Int. Cl. G01N 33/53; C07K 14/00; C12N 15/00

(52) U.S. Cl. 435/7.1; 435/320.1; 530/350; 530/412

(57) ABSTRACT

This application relates to libraries of toxin mutants, and to methods of using same in the development of targeted against specific cell types.

### BACKGROUND OF THE INVENTION

This application relates to libraries of toxin mutants, and to methods of using same in the development of therapeutics targeted against specific cell types.

Plant and bacterial toxins have a structural organization with two or more polypeptide domains or subunits responsible for distinct functions, referred to as A and B. The toxins may be referred to as AB_{X} toxins where x represents the number of identical or homologous B subunits in the toxin. This family of framework-related toxins includes examples such as Shiga and Shiga-like toxins, the E. coli heat-labile enterotoxins, cholera toxin, diphtheria toxin, pertussis toxin, Pseudomonas aeruginosa exotoxin A (Olsnes, S, and Sandvik, K. (1988) in Immunotoxins pp. 39-73, Kluwer Academic, Boston; Sandvik, K., Dubinina, E., Garred, O., et al. (1992) Biochem. Soc. Trans. 20:724) as well as plant toxins such as ricin and abrin. In some cases the toxin are heteromeric, in that the B chains are actually separate entities that connect to the toxic A chain via a non-covalent bonding. In other cases, the toxin is monomeric, since the B chain is part of the same protein when the toxin is produced in nature.

Based on their ability to block protein synthesis, proteins such as Shiga and Shiga-like toxins as well as ricin, abrin, gelonin, crotin, pokeweed antiviral protein, saporin, momordin, modeccin, sarcin, diphtheria toxin and exotoxin A have been referred to as ribosome-inactivating proteins (RIP) The potency of RIPS is exceedingly high; one molecule of diphtheria toxin A chain (Yamaizumi, et al. (1978) Cell 15:245-250) or ricin A chain (Eiklid, et al. (1980) Exp. Cell Res. 126:321-326) having been shown to be sufficient to kill a eukaryotic cell.

International Patent Publication No. WO99/40185 describes libraries of mutant toxins in which mutations are introduced into the binding domain to alter the type of cells to which the toxic species are delivered. The new proteins are derived by mutating a binding subunit of the wild type heteromeric protein cytotoxic protein to create a library of microorganism clones producing mutant proteins, which are then screened for the ability to, specifically bind to and kill a target cell type.

U.S. Pat. No. 5,552,144 discloses a Shigella-like toxin II variant to which a mutation is introduced into the A chain at position 167 to change the amino acid at this position to one with a different charge. This resulted in a toxin with less of the enzymatic activity associated with toxicity.

U.S. Pat. No. 6,593,132 describes recombinant toxic proteins which are specifically toxic to diseased cells but do not depend for their specificity of action on a specific cell binding component. The recombinant proteins of the '132 patent have an A chain of a ricin-like toxin linked to a B chain by a synthetic linker sequence which may be cleaved specifically by a protease localized in cells or tissues affected by a specific disease to liberate the toxic A chain thereby selectively inhibiting or destroying the diseased cells or tissues.

U.S. Pat. No. 6,649,742 disclosed Type I ribosome-inactivating proteins (RIPs) and analogs of the RIPs having a cysteine available for disulfide bonding to targeting molecules. The RIPS and RIP analogs are used as components of cytotoxic therapeutic agents to selectively eliminate any cell type to which the RIP component is targeted by the specific binding capacity of the second component of the agent.

### SUMMARY OF THE INVENTION

The present provides combinatorial protein libraries comprising a plurality of protein species, in which each protein species comprising an A chain of at heteromeric toxic protein into which an insert has been introduced. In accordance with the invention, the insert is a polypeptide of varying amino acid sequence having a length of 2 or more amino acid residues, for example from 3 to 200 amino acid residues; and the insert is introduced into the protease-sensitive loop of the A chain sequence. The result of the introduction of the insert creates an artificial binding domain within the A chain, such that the A chain develops toxic specificity which is independent of and different from the normal specificity associated with the B chains binding domains. Screening of the library allows the selection and identification of mutant toxins that are specific for different cell types, including cancer cell types. In one embodiment of the invention, the combinatorial library comprises protein species that are formed by introducing the insert into a Shiga-like toxin I A chain, for example in the region between amino acids 242 and 261, as defined with reference to Seq. ID No. 1.

The invention also provides a combinatorial expression library comprising a plurality of species of expression systems, each species expressing a protein species comprising an A chain of a heteromeric toxic protein into which the insert as described above has been introduced. Expression of protein from the combinatorial expression library results in the formation of a combinatorial protein library.

In a further aspect, the invention provides a composition for treating melanoma, and methods for using such a composition. The composition comprises a protein species comprising an A chain of a heteromeric toxic protein into which a polypeptide having a length of 2 or more amino acid residues, for example from 3 to 200 amino acid residues, is introduced into the protease-sensitive loop of the A chain sequence. The insert is selected such that protein species has toxic activity towards melanoma cells. The protein species is used in the treatment of melanoma by administering it to a patient diagnosed with melanoma, in an amount sufficient to result in the reduction of the number of living melanoma cells.

In a further aspect, the invention provides a composition for treating other types of cancer. The composition comprises a protein species comprising an A chain of a heteromeric toxic protein into which a polypeptide having a length of 2 or more amino acid residues, for example from 3 to 200 amino acid residues, is introduced into the protease-sensitive loop of the A chain sequence. The insert is selected such that protein species has toxic activity towards the cancer cells. In a specific embodiment, the insert is selected to bind to MUC-1 receptors. The protein species is used in the treatment of melanoma by administering it to a patient diagnosed with melanoma, in an amount sufficient to result in the reduction of the number of living melanoma cells.

In a further aspect, the invention provides a method for identifying ligands that bind to specific targets/receptors, such as tumor markers known to exist on cancer cells. In his method, the toxin of the combinatorial library serves as a reporter, and a combinatorial protein library in accordance with the invention is screened against cells known to possess the target/receptor. Proteins which are shown to be toxic to the cells are evaluated to determine the sequence of the inserted region. Peptides of this sequence can then be used, in combination with a toxin or other molecules, to direct compounds to cells possessing the target/receptor.

In yet a further aspect of the invention, a method is provided for identifying toxic substances specific for a known cell marker. In this embodiment of the invention, the toxin need not serve as reporter. Thus, cells having the marker, or an isolated target/receptor, where available, are exposed to the combinatorial protein library. In preferred embodiments, the cells or the isolated target/receptor are immobilized on a solid support, such as in plastic wells. Captured proteins from the library are then rescreened against cells to confirm their toxicity and specificity for cells expressing the target/receptor, and their suitability for use as a therapeutic.

### BRIEF DESCRIPTION OF THE DRAWINGS

FIG. 1 is a schematic diagram depicting the A1 and A2 domains of SLT-1. The A chain is composed of 293 amino acids. The chain is cleaved by furin to produce an A1 catalytic fragment and an A2 C-terminal tail non-covalently associated with the B pentamer. A protease-sensitive loop (stripped area) is defined by the only two cysteine residues in the A chain (Cys 242 and 261). Tyr77, Glu167, Arg170 and Trp203 represent residues crucial for the catalytic activity of the A1 domain (arrows).

FIG. 2A is a schematic representation of the SLT-1 A chain (1-293) with the breast cancer-associated MUC1 epitope PDTRPAP (control sequence recognized by the mAb One M27) inserted between residues 245 and 246 and a 6-Histidine tag at its N-terminus.

FIG. 2B is a depiction of our SLT-1 A chain-tripeptide library constriction where the three key positions of the MUC1 epitope recognized by the mAb One M27 were randomized (XXX region). The tripeptide library was inserted in a naturally occurring loop region of the A chain created by the presence of a disulfide bridge between Cys 242 and Cys 261.

FIG. 3 shows a representative ELISA data set from screening 96 distinct single A chain variants from our SLT-1 A chain-tripeptide library with in mAb One M27. Toxin variant #41 (Tables 1 and 2) gave a strong ELISA signal and had the expected epitope.

FIG. 4 shows a schematic diagram of a 7-amino acid random segment inserted between residues 245 and 246 of the A chain.

FIG. 5 shows the results of tests on seven toxin variants that: were identified as repeatable killers of the human melanoma cell line 518A2The abscissa represents the log concentration of toxin used to treat the cells and the ordinate depicts the observed percentage of cells that are viable after 48 hours. The closed triangles depicts the effect of the wild type toxin on 518A2 cells while the two most efficacious A chain variants were termed SAM#3 (open squares) and SAM#5 (X symbols).

### DETAILED DESCRIPTION OF THE INVENTION

The present invention relates to a combinatorial protein library comprising a plurality of protein species, each protein species comprising an A chain of a heteromeric toxic protein into which an insert has been introduced. The insert is a polypeptide of varying amino acid sequence having a length of 2 or more amino acid residues, for example from 3 to 200 amino acid residues; and is introduced into the protease-sensitive loop of the A chain sequence. The library provides a collection of protein species that can be screened for individual proteins that are toxic against specific cell types, such as specific cancer cell types. Individual protein species thus selected are suitably used in the treatment of the cancer.

As used in the specification and claims of this application, the term “combinatorial library” refers to a mixture of species each of which has a common portion and a variable portion. In the case of a “combinatorial protein library” each of the species is a protein or peptide, and the common portions and the variable portions are each amino acid sequences. In the case of a combinatorial expression library, the species are microorganisms, expression vectors or polynucleotides which, when expressed, produce proteins or peptides having common portions and variable portions. In this case, the common portions and the variable portions are each nucleotide sequences. Since the purpose of the combinatorial library is to provide multiple variants for screening purposes, the combinatorial library preferably contains at least 100 distinct species of protein or expression unit, more preferably at least 1000 distinct species.

As used in the specification and claims of this application, the term “heteromeric toxic protein” refers to the class of protein toxins with the common organization theme of being heteromeric in nature with two or more polypeptide domains or subunits responsible for distinct functions (Merritt, E. A., and Hol, W. G. J. (1995) Curr. Opin. Struct. Biol. 5:1651). In such proteins, the two or more subunits or domains could be referred to as A and B, and the toxins as AB_{X} toxins where x represents the number of identical or homologous B subunits in the toxin. This family of framework-related toxins includes examples such as Shiga and Shiga-like toxins, the E. coli heat-labile enterotoxins, cholera toxin, diphtheria toxin, pertussis toxin, Pseudomonas aeruginosa exotoxin A as well as plant toxins such as ricin and abrin. Based on their ability to block protein synthesis, proteins such as Shiga and Shiga-like toxins as well as ricin, abrin, gelonin, crotin, pokeweed antiviral protein, saporin, momordin, modeccin, sarcin, diphtheria toxin and exotoxin A have been referred to as ribosome-inactivating proteins (RIP). In these naturally-occurring heteromeric toxic proteins, the A chain is the toxic portion, while the B chains form a binding moiety which binds to a receptor on a cell susceptible to the toxin, thereby delivering the A chain to the cell.

One specific example of the A chain of a heteromeric toxic protein is the A chain of SLT-1 which has the sequence given is Seq. ID No, 1. The A chain of SLT-1 comprises of 293 amino acids with the enzymatic (toxic) domain spanning residues 1 to 239. A protease sensitive loop encompassing residues 242 to 261 is normally exposed, and is a suitable site for inserting a peptide sequence.

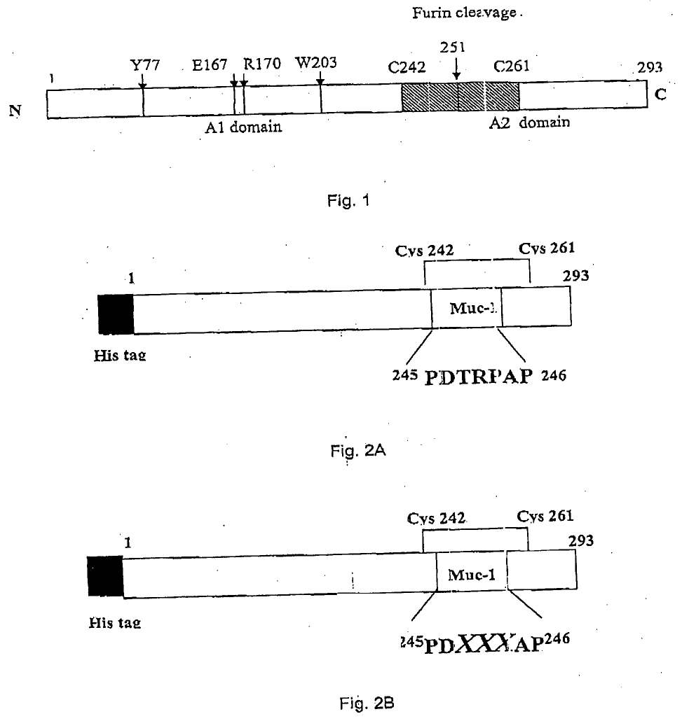

SLT-1 is a type II ribosome inactivating protein produced by pathogenic strain of Escherichia coli (0157:H7) (24). SLT-1 is an AB5 complex of about 70 kD (O'Brien, A. D., and Holmnes, R. K. (1987) Shiga and Shiga-like toxins. Microbiol. Rev 51, 206-220). The single 32 kD catalytic A subunit is non-covalently associated with a pentamer of five identical 7.7 kD B subunits. The B subunit pentamer recognizes the glycolipid globotriaosylceramide (also known as CD77 or Gb3) on the surface of target cells (Lingwood, C. A. (1993) Verotoxins and their glycolipid receptors. Adv Lipid Res 25, 189-211; Jacewicz, et al. (1986) Pathogenesis of shigella diarrhea). XI Isolation of a shigella toxin-binding glycolipid from rabbit jejunum and HeLa cells and its identification as globotriaosylceramide. J Exp Med 163, 1391-1404). A protease-sensitive loop located between Cys242 and 261 at the C terminus of the A chain is cleaved by furin during cellular routing (FIG. 1). The A chain remains associated with its B subunit pentamer due to an intrachain disulfide bond between Cys242 and Cys261 as it travels to the ER lumen (Sandvig., et al. (989) Endocytosis from coated pits of Shiga toxin: a glycolipid-binding protein from Shigella dysenteriae 1. J Cell Biol 108, 1331-1343;32. Garred, et al. (1995) Role of processing and intracellular transport for optimal toxicity of Shiga toxin and toxin mutants. Exp Cell Res; 218, 39-49). The disulfide bond is finally reduced in the ER lumen and the A1 chain (first 251 aa) is released and subsequently retrotranslocated to the cytosol where it inactivates ribosomes (O'Brien, et al. (1992) Shiga toxin: biochemistry, genetics, mode of action, and role in pathogenesis. Curr Top Microbiol Immunol 180, 65-94). More specifically, the A chain of SLT-1 is a N-glycosidase that catalytically cleaves a specific adenine nucleotide (4324) from the 28 S rRNA (Brigotti, et al. (1997) The RNA-N-glycosidase activity of Shiga-like toxin I: kinetic parameters of the native and activated toxin. Toxicon 35, 1431-1437). This event leads to the inhibition of protein synthesis by preventing the binding of aminoacyl tRNAs to the ribosome and halting protein elongation. Mutagenesis studies as well as structural analysis performed on the A chains of ST and ricin have delineated key conserved residues involved in catalytic activity (Deresiewicz, et al.(1992) Mutations affecting the activity of the Shiga-like toxin I A-chain. Biochemistry 31, 3.272-3280; Ready, et al. (1991) Site-directed mutagenesis of ricin A-chain and implications for the mechanism of action. Proteins 10, 270-278). Residues crucial for catalytic activity of SLT-1 are tyrosine 77, glutamic acid 167, arginine 170 and tryptophan 203 (Hovde, et al. (1988) Evidence that glutamic acid 167 is an active-site residue of Shiga-like toxin I. Proc Natl Acad Sci USA. 85, 2568-2572; Yamasaki, et al. (1991) Importance of arginine at position 170 of the A subunit of Vero toxin I produced by enterohemorrhagic Escherichia coli for toxin activity. Microb Pathog 11, 1-9). In addition, binding of the toxin to the cell surface is critical to introduction into the cell and thus for toxic activity. Because of this the A chain alone is not significantly toxic.

In addition to the A chain of SLT-1, other toxins may also be used to form libraries and compositions in accordance with the invention, and may be used in the methods of the invention. Specifically, Shiga and other Shiga-like toxins, as well as ricin, abrin, gelonin, crotin, pokeweed antiviral protein, saporin, momordin, modeccin, sarcin, diphtheria toxin and exotoxin A, and other functionally-related ribosome inactivating proteins (RIP) may be used.

For purposes of making the combinatorial library of the invention, short amino acid sequences of at least 2 amino acids, for example of 3 to 200 amino acids residues in length, are inserted into this protease sensitive loop. The number of amino acids in the insert defines the number of possible random variants that can be in the library. For example, when the number of amino acids in the insert is 3, the maximum number of variants is 20³ or 8000 variants. Larger inserts provide corresponding larger-numbers of possible variants.

As an alternative to using inserts with purely random sequences, inserts can be designed based on a known template. For example, as described below in the context of Muc-1, variations in a sequence known to provide receptor binding properties for a particular cell type can be used to identify an insert that provides for optimization of the toxic properties of the protein construct. This same optimization may be performed on an individual sequence isolated by screening of a larger combinatorial library. It will be appreciated, however, that the insert in the proof of principle tests of Example 1 use an insert which is the target/receptor, while in the actual case the insert would be, based on the sequence of a known ligand to be optimized for maximum effectiveness and specificity.

This approach, in which a specific, known receptor type is targeted illustrates a further aspect of the invention, namely a method for identifying peptide ligands that bind to specific targets/receptors; such as tumor markers known to exist on cancer cells. In this method, a combinatorial protein library in accordance with the invention is screened against cells known to possess the target/receptor. The toxin serves as the reporter, such that proteins which are shown to be toxic to the tells are evaluated to determine the sequence of the inserted region. Peptides of this sequence can then be used, in combination with a toxin or other molecules, to direct compounds to cells possessing the target/receptor. Further testing peptides of the sequence of the inserted region may be appropriate to confirm that: they are a ligand for the specific target/receptor, as opposed to some other receptor on the cell type. This can be done using binding assays with isolated receptor, where such are available.

The invention also provides a method for identifying toxic substances specific for a known cell marker, and particularly markers that are available in isolated form. In this embodiment of the invention, the toxin need not serve as reporter. Thus, cells having the marker, or an isolated target/receptor, where available, are exposed to the combinatorial protein library. In preferred embodiments, the cells or the isolated target/receptor are immobilized oil a solid support, such as in plastic wells. Captured proteins from the library are then rescreened against cells to confirm their toxicity and specificity for cells expressing the target/receptor, and their suitability for use as a therapeutic. This method can be used to identify toxins with binding inserts specific for any tumor marker or cell receptor, including without limitation tumor markers such as mucins such as MUC-1 and its glycoforms, Her-2, Her2-Neu, tyrosine kinase markers, EGER, GD2, and GD3.

Thus, in accordance with this specific aspect of the invention, a method for isolating a toxin specific for a known target/receptor is provided that comprises the steps of:

(a) exposing the target/receptor to a combinatorial protein library comprising a plurality of protein species, each protein species comprising an A chain of a toxic protein into which an insert has been introduced, wherein,
the insert is a polypeptide of varying amino acid sequence having a length of least 2 amino acid residues; and
the insert is introduced into the protease-sensitive Loop of the A chain sequence; and

(b) isolating at least one protein species from the combinatorial protein library captured by binding to the target/receptor. As used in the specification and claims hereof, the term “isolating” refers to any mechanism for obtaining a composition containing the protein separated from the milieu in which it is expressed in a form suitable for further analysis. This would include release from the target/receptor following capture (for example by exposure to a competitive binding agent) or isolation from a culture of a clone expressing the protein found to be captured. The method may further comprises the step of screening the isolated protein against cells expressing the target/receptor, to confirm their toxicity for cells expressing the target/receptor. Procedures suitable for this screening are described in Example 3. As noted above, in this method, the target/receptor may be a purified target/receptor and may be immobilized on pa solid support. The target/receptor may also be on the surface of cells, which may be immobilized. Where the target/receptor is on the surface of cells, the toxin can serve as a reporter, and the death of the cells is indicative of receptor binding.

Our experience with constructing SLT-1 libraries has pointed out a number of practical issues that are appropriately considered in selecting a protein template for building combinatorial libraries. An important factor is to choose a single chain protein, preferably a bacterial protein of less than 300 amino acids: (if the libraries are to be expressed in prokaryotes). Use of a smaller toxin both increases its potential to penetrate into solid tumors and reduces its immunogenicity. Secondly, the protein template should spontaneously fold in solution into its active form such that there is minimal need for host's chaperones. One should avoid for example scaffolds that contain multiple cysteine residues normally involved in disulfide bridges. In addition, a single chain protein as opposed to a multi-subunit complex may be more easily exported from bacteria. Thirdly, the protein template should possess an enzymatic activity, which can rapidly be measured to confirm the proper folding of peptide variants containing single and multiple site-directed mutations. Fourthly, a simple screening approach should be incorporated into the design of combinatorial libraries. Such searches should be amenable to high-throughput screening methods. The catalytic A chain of SLT-1 (residues 1 to 293) meets these criteria because it is a single chain that lacks any known receptor binding function, and that has a well defined structure and catalytic site.

A further aspect of the invention is a combinatorial expression library comprising a plurality of species of expression systems. Each species within the expression library expresses a protein species comprising an A chin of a heteromeric toxic protein into which an insert has been introduced. The insert is a polypeptide of varying amino acid sequence having a length of 2 or more amino acid residues, for example from 3 to 200 amino acid residues; and is introduced into the protease-sensitive loop of the A chain sequence, Suitable expression systems include plasmids and viral vectors.

The invention will now be further described with reference to the following, non-limiting examples.

### EXAMPLE 1

### Proof-of-Concept: Design and Mining of a Prototypic A Chain-Tripeptide Library

We originally created a simple tripeptide library inserted in the C-terminal protease-sensitive loop of the SLT-1 A chain (FIGS. 2A and B). This A chain loop region is naturally constrained due to the presence of a single disulfide bond bridging Cys242 to Cys261. The maximal diversity of this library can thus be calculated to be 20³ or 8000 permutations of a tripeptide sequence. As a proof-of-concept that A chain libraries can easily be screened for a new receptor-binding activity, we picked more than 3000 colonies from this A chain-tripeptide library and purified the mutant toxin produced by each clone. We noticed very early in this study that the level of expression of A chain mutant was dramatically increased when expressed in the presence of the wild-type SLT-1 B subunit. Thus the mutant forms of A chain were expressed and initially-purified as AB5 toxin variants. Since all A subunits harbor a polyHis purification tag, it is relatively easy to remove the B subunit with denaturants (urea for example) while recovering the A chain on metal-affinity columns or beads. Western blots performed on randomly selected bacterial clones indicated that >70% of these colonies produced significant amounts of these A chain mutants.

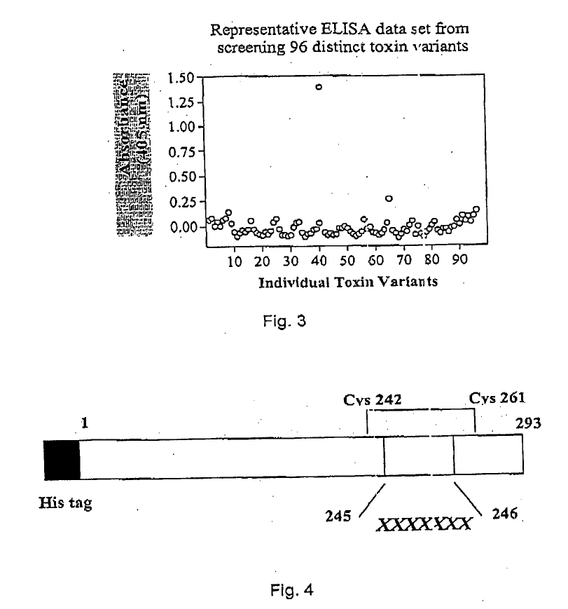

These toxin variants were then coated in individual wells of 96-well plates and screened by ELISA for their ability to bind the monoclonal antibody One M27 (Linsley, et al.(1988) Monoclonal antibodies reactive with mucin glycoproteins found in sera from breast cancer patients. Cancer Res 48, 2138-2148), directed at the well-characterized breast cancer tripeptide epitope Thr-Arg-Pro of the human MUC1 tandem repeat (Gendler, et al. (1988) A highly immunogenic region of a human polymorphic epithelial mucin expressed by carcinomas is made up of tandem repeats. J Biol Chem 263, 12821)-12823; Girling, et al. (1989) A core protein epitope of the polymorphic epithelial mucin detected by the monoclonal antibody SM-3 is selectively exposed in a range of primary carcinomas. Int J Cancer 43, 1072-1076). As shown in Tables 1 and 2, most A chain mutants neither coded for the tripeptide insert that matched the targeted epitope of One M27 nor were recognized by the antibody. However on two occasions, a toxin variant harboring the exact epitope (Table 1, FIG. 3) gave a strong ELISA signal comparable to the one observed with our control A chain harboring the MUC1 tripeptide epitope and had the expected epitope sequence in its randomized tripeptide region. A typical ELISA data set for 96 A chain mutants is presented in FIG. 3, highlighting the fact that the majority of A chain mutants did not recognized the mAb One M27 except for an A chain variant (mutant #41 in Tables 1 and 2). These results clearly established that A chain libraries can easily be constructed and screened to find toxin variants able to specifically target a given receptor, in this case an antigen-combining site.

### EXAMPLE 2

### Making of Combinatorial SLT-1 A-Heptapeptide Library

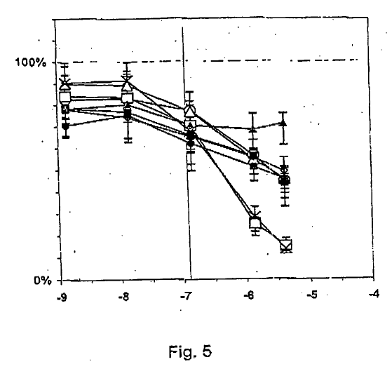

Library diversity represents a crucial parameter in screening, combinatorial libraries for ligands able to bind specifically and with high affinity to a particular target. The SLT-1 A chain-tripeptide library described in Example 1 has a maximal diversity of 20³ or 8000 possible mutated A chains. This sampling repertoire is small but was useful in mapping a tripeptide epitope to establish our proof-of-concept. A seven-residue library (20⁷ or 1.3×10⁹ possible mutants) represents a more typical minimal diversity level commonly used in designing phage display as well as synthetic peptide libraries. Thus, as a starting point, we built a SLT-1 A chain library with a 7-amino acid long random sequence inserted in its C-terminus. This library was installed in the A chain's protease sensitive loop, a loop region naturally constrained by a disulfide bond. This library pro-vided sufficient diversity to insure that A chain toxin variants can be identified that target new or known internalized receptors on cancer cells. All elements of this library-(as well as all other libraries proposed) contain a N-terminal His tag to quickly purify A chain mutants. The library (FIG. 4) was generated using a megaprimer PCR strategy (Sarkar G, and Sommers S, (1990). The ‘megaprimer’ method of site-directed mutagenesis. Biotechniques 8, 404-407). The megaprimer strategy is widely used to introduce mutations into target DNA sequence, by way of two rounds of PCR that use two flanking primers and an internal mutagenic primer. A description of the library design for the SLT-1 A chain-heptapeptide library will serve as the example for other future single A chain libraries. The SLT-1 A chain-heptapeptide library (FIG. 5) harbors a 7-amino acid random insertion between amino acid 245 and 246 of the A chain, the identical site used to construct our SLT-1 A chain-tripeptide library (Tables 1, 2, FIG. 3). Briefly, two flanking primers A (GTT ACT GTG ACA GCT GAA GCT TTA CGT TTA CG (Seq. ID No. 2) and B (GAG AAG AAG AGA CTG CAG ATT CCA TCT GTT G (Seq. ID No. 3)) carrying Hind111 and Pst1 restriction sites respectively were annealed within the 5′ and 3′ ends of the SLT-1 operon. A library oligonucleotide F containing all seven random amino acid (NNS) as well as a long matching sequence to anneal to the template were synthesized. In the synthesis of the random oligonucleotide, the relative representation of each amino acid was improved by restricting the third position of each codon to G or T (Noren, K. A., and Noren, C. J. (2001) Construction of high-complexity combinatorial phage display peptide libraries. Methods 23, 169-178). This type of restriction reduces the overall DNA sequence complexity as well as coding discrepancy between residues (Reidhaar-Olson, et al. (1991) Random mutagenesis of protein sequences using oligonucleotide cassettes. Methods Enzymo 208, 564-586). This strategy also minimizes the occurrence of stop codons (TAA and TGA while the stop codon (TAG) is suppressed by using a supE bacterial strain, which specifies for the insertion of a Gln residue when TAG codons are translated. The first PCR reaction was performed using primers A and F and the resulting product purified by denaturing polyacrylamide gel electrophoresis. This product then served as the megaprimer with primer B for a second PCR reaction to amplify the random DNA. The final library DNA (PCR product) will then be digested with Hind111 and Pst1 and cloned into the backbone of a pECHE9a expression vector (MTI, Toronto). The E. coli strain JM101 was subsequently transformed with the resulting pECHE vector and single bacterial colonies picked, lysed and their supernatants analyzed for the expression of single A chain toxins or layered on cancer cells and screened using the SRB for cell cytotoxicity assay.

### EXAMPLE 3

### Mining the Combinatorial SLT-1 A-Heptapeptide Library Against Cancer Cell Lines Using a Cytotoxicity Assay

We screened our SLT-1 A-heptapeptide library using the cytotoxic function of the A chain as a reporter signal. Cytotoxicity is a more informative property to measure than binding to a receptor since it implies that the toxin is internalized, processed and delivered near ribosomes, clearly a multi-step event. The cytotoxicity assay was essentially performed as previously described (Bray, et al. (2001) Probing the surface of eukaryotic cells using combinatorial toxin libraries. Current Biology 11, 697-701). Briefly, the strategy to screen all our A chain libraries was based on the following principles. Established cancer cell lines such as SK-BR-3 Unman breast), CAMA-1 (human breast), 518A2 (human melanoma), PC3 (human prostate) and B16 (murine melanoma) were grown in 96-well plates and used as targets in the primary screen stages. These cell lines were initially selected for our holotoxin library searches (Bray, supra) based on their adherence (plastic), their cell viability staining properties (SRB) in a high-throughput screening setting as well as their lack of receptor and sensitivity to native SLT-1 (to insure a reduced level of false positives). Single bacterial colonies from each library were picked and grown in 96 deep well plates The cells were harvested, lysed, and their lysates clarified. Since all ex pressed SLT-1 A chain variants have a 6 histidine tag at their N-terminus, each of them was purified from their lysate using nickel-affinity beads (96-well format) and layered on target cells. The plates containing the target cells treated with A chain variants were then incubate d at 37° C. for 48 hours, followed by fixation and staining with Sulforhodimine B (SRB). The SRB assay is a colorimetric end-point assay, which quantifies viable cells by staining their cellular protein content (Skehan, et al. (1990) New colormetric cytotoxicity assay for anticancer-drug screening. J Natl Cancer Inst 82, 1107-1112). The SRB assay has been adopted by NCI/NIH for their high-throughput screening of drug candidates on cancer cell lines. Viability assays were repeated for any bacterial extracts leading to cell death. A first round of screening was performed on more than 5000 bacterial clones (equivalent to 5000 distinct A chain toxins) and 7 toxin variants were identified as repeatable killers of the human melanoma cell line 518A2 (FIG. 5). The abscissa represents the log concentration of toxin used to treat the cells and the ordinate depicts the observed percentage of cells that are viable after 48 hours. The closed triangles depicts the effect of the wild type toxin on 518A2 cells while the two most efficacious A chain variants were termed SAM#3 (open squares) and SAM#5 (X symbols).

Seven promising A chain variants were then re-screened against a panel of cell lines (Vero [Monkey, normal kidney]; PC-3 [Human, prostate cancer]; HepG2 [Human, hepatoma]; SiHa [Human, cervical cancer]; PanC [Human, pancreatic cancer]; SKBR-3 [Human, breast cancer]; 518-A2 [Human, melanoma]; U87 [Human, glioma]; B16-F10 [Mouse, melanoma]; HS-216 [Human, normal fibroblast]; CAMA-1 [Human, breast cancer]; OVCar-3 [Human, ovarian cancer]). Of these seven four were observed to observed to have activity against a cancer cell line, two for 518-A2 human melanoma, and one each for SiHa (human cervical cancer cells) and U87-A (human brain cancer cells; glioma).

The genes coding for the two A chain toxins (SAM3 and SAM5) that resulted in toxicity of the human melanoma cell lines were sequenced to determine the amino acid sequences inserted between residues 245 and 246 of the wild-type A chain. The sequences, including the His-tag, are listed in Seq. ID Nos 4 and 5, respectively.

TABLE 1

DNA sequences of randomly picked clones from
the SLT-1 A chain-tripeptide library. Mutated
bases in bold characters. Mutant #41 was
identified in our ELISA screen as a strong
binder of mAb Onc M27 (FIG. 3).

| | | Diversity |
|---|---|---|
| | | (nucleotide |
| SLT-1 A | | changes in |
| chain | | mutated |
| variant | Nucleotide sequence | region) |
| MUC1 | CCA GAC ACG CGA CCA GCT CCA | 0/9 |
| epitope | | |
| Mutant #1 | CCA GAC GGG ATC GGG GCT CCA | 8/9 |
| Mutant #2 | CCA GAC CTG GAG ATG GCT CCA | 8/9 |
| Mutant #3 | CCA GAC CCC CGT GGG GCT CCA | 6/9 |
| Mutant #4 | CCA GAC GAC GAC TTG GCT CCA | 9/9 |
| Mutant #5 | CCA GAC GTC CGG TGG GCT CCA | 7/9 |
| Mutant #6 | CCA GAC CAG CGC TGG GCT CCA | 6/9 |
| Mutant #7 | CCA GAC CTC AGG ATG GCT CCA | 8/9 |
| Mutant #8 | CCA GAC TCC CAG GAG GCT CCA | 7/9 |
| Mutant #9 | CCA GAC TCC GAC CCC GCT CCA | 6/9 |
| Mutant #41 | CCA GAC ACG CGC CCC GCT CCA | 2/9 |

TABLE 2

Amino acid sequence alignment of randomly
selected clones from the SLT-1 A chain-
tripeptide library and ELISA signal of purified
SLT-1 A chain variants detected with a mAb
(Onc M27) raised against the MUC1 epitope Thr-
Arg-Pro. Mutated tripeptide region in bold
characters. Mutant #41 was identified in our
ELISA screen as a strong binder of mAb Onc M27.

| | | |
|---|---|---|
| SLT-1 A | | ELISA |
| chain | | readings |
| variant | Deduced amino acid sequence | (405 nm) |

| | | |
|---|---|---|
| MUC1 | CHHHPD TRP APASRVARMASDEFPSMC | 1.3 |
| epitope | | |
| Mutant #1 | CHHHPD GIG APASRVARMASDEFPSMC | 0.08 |
| Mutant #2 | CHHHPD LQM APASRVARMASDEFPSMC | 0.03 |
| Mutant #3 | CHHHPD PRG APASRVARMASDEFPSMC | 0.03 |
| Mutant #4 | CHHHPD DDL APASRVARMASDEFPSMC | 0.06 |
| Mutant #5 | CHHHPD VRW APASRVARMASDEFPSMC | 0.07 |
| Mutant #6 | CHHHPD QRL APASRVARMASDEFPSMC | 0.06 |
| Mutant #7 | CHHHPD LRM APASRVARMASDEFPSMC | 0.11 |
| Mutant #8 | CHHHPD SQE APASRVARMASDEFPSMC | 0.13 |
| Mutant #9 | CHHHPD SDP APASRVARMASDEFPSMC | 0.07 |
| Mutant #41 | CHHHPD TRP APASRVARMASDEFPSMC | 1.25 |

1. A combinatorial protein library comprising a plurality of protein species each protein species comprising an A chain of a toxic protein into which an insert has been introduced, wherein,
(a) the insert is a polypeptide of varying amino acid sequence halving a length of at least 2 amino acid residues; and
(b) the insert is introduced into the protease-sensitive loop of the A chain sequence.

2. The combinatorial protein library of claim 1, wherein the Horary comprises at least 100 protein species.

3. The combinatorial protein library of claim 1, wherein the protein species are formed by introducing the insert into a Shiga-like toxin I A chain.

4. The combinatorial protein library of claim 3, wherein the protein species is formed by introducing the insert between amino acids 242 and 261, as defined with reference to Seq. ID No. 1.

5. The combinatorial protein library of claim 4, wherein the protein species is formed by introducing the insert between amino acids 245 and 246, as defined with reference to Seq. ID No. 1.

6. The combinatorial protein library of claim 3, wherein the protein species is formed by introducing the insert before or after amino acids 1-239 of the Shiga-like toxin I A chain, as defined with reference to Seq. ID No. 1.

7. The combinatorial protein library of claim 1, wherein the insert has a length of 7 amino acids.

8. A combinatorial expression library comprising a plurality of species of expression systems, each species expressing a protein species in accordance with claim 1.

9. A mutant protein comprising an A chain of a toxic protein into which an insert has been introduced, wherein,
(a) the insert is a polypeptide of varying, amino acid sequence having a length of at least 2 amino acid residues; and
(b) the insert is introduced into the protease-sensitive loop of the A chain sequence.

10. The mutant protein of claim 9, wherein the A chain of a toxic protein is a Shiga-like toxin I A chain.

11. The mutant protein of claim 10, wherein the insert in introduced between amino acids 242 and 261, as defined with reference to Seq. ID No. 1.

12. The mutant protein of claim 11 wherein the insert is introduced between amino acids 245 and 246, as defined with reference to Seq. ID No. 1.

13. The mutant protein of claim 12, wherein the insert comprises the sequence IYSNKLM (Seq. ID No. 6).

14. The mutant protein of claim 12, wherein the insert comprises the sequence AAFADLI (Seq. ID No. 7).

15. The mutant protein of claim 10, wherein the insert is introduced before or after amino acids 1-239 of the Shiga-like toxin I A chain, as defined with reference to Seq. ID No. 1.

16. The mutant protein of claim 9, wherein the insert has a length of 7 amino acids.

17. A method for identifying a ligand that bind to a specific target/receptor, comprising the steps of:
(a) exposing cells known to possess the target/receptor to members of a combinatorial protein library in accordance with claim 1;
(b) selecting members of the protein library which are observed to be toxic to the cells;
(c) evaluating the selected members of the protein library to determine the sequence of the inserted region, whereby; a peptide of the sequence of the inserted region is identified as a possible ligand for a target/receptor on the cell; and
(d) further testing peptides of the sequence of the inserted region to confirm that they are a ligand for the specific target/receptor.

18. A method for isolating a toxin specific for a known target/receptor comprising the steps of:
(a) exposing the target/receptor to a combinatorial protein library according to claim 1; and
(b) isolating at least one protein species from the combinatorial protein library captured by binding to the target/receptor.

19. The method of claim 18, further comprising the step of screening the isolated protein against cells expressing the target/receptor, to confirm their toxicity for cells expressing the target/receptor.

20. The method of claim 18, wherein the target/receptor is a purified target/receptor and is immobilized on a solid support.

21. The method of claim 20, wherein the target/receptor is on the surface of cells.

22. The method of claim 21, wherein the cells are immobilized on a solid support.

23. The method of claim 21, wherein the toxin serves as a reporter, and the death of the cells is indicative of receptor binding.
